(19) United States
(12) Patent Application Publication
(10) Pub. No.: US 2013/0236922 A1
(43) Pub. Date: Sep. 12, 2013

(54) COPPER(I)-ION SELECTIVE FLUORESCENT PROBE, METHOD FOR PREPARING THE SAME, METHOD FOR DIAGNOSING MALIGNANT DISEASE AND DIAGNOSIS KIT USING THE PROBE

(71) Applicants: Bong-Rae CHO, Seoul (KR); Hoon-Jai CHUN, Seoul (KR)

(72) Inventors: Bong-Rae CHO, Seoul (KR); Hoon-Jai CHUN, Seoul (KR)

(21) Appl. No.: 13/476,163

(22) Filed: May 21, 2012

(30) Foreign Application Priority Data
Oct. 12, 2011 (KR) 10-2011-0104150
Feb. 16, 2012 (KR) 10-2012-0015602

(51) Int. Cl. G01N 21/64; C07C 323/25

(52) U.S. Cl. CPC G01N 21/6486; C07C 323/25
USPC 435/29; 564/180

(57) ABSTRACT

The present disclosure relates to a copper (I) ion-selective fluorescent probe, a method for preparing the same, and a method for diagnosing malignant disease and a diagnosis kit using the probe. The fluorescent probe according to the present disclosure is capable of detecting free copper (I) ions inside cells for a long time with high selectivity and sensitivity for copper (I) ion, with a penetration depth longer than 90 μm in living cells and tissues and without the problems of mistargeting and photobleaching. Accordingly, since a biological sample can be imaged for a long period of time with high resolution without damage, presence of malignance disease in the target biological sample can be diagnosed faster, more accurately and more easily.

### CROSS-REFERENCE TO RELATED APPLICATIONS

This application claims priority under 35 U.S.C. §119 to Korean Patent Application Nos. 10-2011-0104150 and 10-2012-0015602, filed on Oct. 12, 2011 and Feb. 16, 2012, respectively, in the Korean Intellectual Property Office, the disclosure of which is incorporated herein by reference in its entirety.

### TECHNICAL FIELD

The present disclosure relates to a copper(I) ion-selective fluorescent probe, a method for preparing the same, and a method for diagnosing malignant disease and a diagnosis kit using the probe.

### BACKGROUND

Copper ion, existing either as oxidized copper(II) ion (Cu²⁺) or as reduced copper(I) ion (Cu¹⁺), is the essential probing metal found in the organs of living organisms. It acts as cofactor in many reactions by cytoplasmic enzymes, mitochondrial enzymes and membrane-oxidizing enzymes. Especially, the level of free copper ion in cells is regulated elaborately. It is because abnormality in the regulation of copper ion level may result in severe diseases such as Menkes disease and Wilson's disease.

Numerous one-photon (OP) fluorescent probes have been developed to understand the role of copper ion in cells (M. Royzen, Z. Dai and J. W. Canary, J. Am. Chem. Soc., 2005, 127, 1612; G. K. Li, Z. Xu, X. C. F. Chen and Z. T. Huang, Chem. Commun., 2008, 1774; H. S. Jung, P. S. Kwon, J. W. Lee, J. I. Kim, C. S. Hong, J. W. Kim, S. Yan, J. Y. Lee, J. H. Lee, T. Joo and J. S. Kim, J. Am. Chem. Soc., 2009, 131, 2008). However, most of the OP fluorescent probes are selective only to the copper(II) ion, and only a few OP fluorescent probes such as pyrazoline- or BODIPY-based dye, (1,4,7,10-tetrathia-13-aza)-15-crown-5 or bis{2-[2-(2-ethylthio)ethylthio]ethyl}amine (BETA) have copper(I) ion selectivity (L. Yang, R. McRae, M. M. Henary, R. Patel, B. Lai, S. Vogt and C. J. Fahrni, Proc. Natl. Acad. Sci. U.S.A., 2005, 102, 11179; L. Zeng, E. W. Miller, A. Pralle, E. Y. Isacoff and C. J. Chang, J. Am. Chem. Soc., 2006, 128, 10; A. F. Chaudhry, V. M. T. Morgan, M. M. Henary, N. Siegel, J. M. Hales, J. W. Perry and C. J. Fahrni, J. Am. Chem. Soc., 2010, 132, 737; D. W. Domaille, L. Zeng and C. J. Chang, J. Am. Chem. Soc., 2010, 132, 1194). Moreover, in order to use the OP fluorescent probes for one-photon microscopy (OPM), excitation by short-wavelength (350-500 μm) light is necessary, which limits penetration depth and causes photobleaching, photodamage, cellular autofluorescence, etc. resulting in restricted applications in tissue imaging.

Accordingly, use of two-photon microscopy (TPM) is essential for detection of copper ion present deep in the living tissue. TPM, wherein two near-infrared photons are used as excitation source, has many advantages over OPM. In particular, penetration depth is increased (>500 μm), localized excitation is possible, and long-term tissue imaging is possible because photodamage and photobleaching are reduced (W. R. Zipfel, R. M. Williams and W. W. Webb, Nat. Biotechnol., 2003, 21, 1369; F. Helmchen and W. Denk, Nat. Methods, 2005, 2, 932). However, since there is no dye available for detection of copper(I) ion by two-photon microscopy, development thereof is urgently needed.

Cu(I) and Zn(II) are cofactors of superoxide dismutase (SOD) antioxidant enzymes and change in the level of the metal ions may be related with neoplasia and malignant disease. From some researches, it was found out that the level of Cu(I) is increased in various malignant diseases such as breast cancer (Kuo H W, Chen S F, Wu C C, et al. Serum and tissue trace elements in patients with breast cancer in Taiwan. Biol Trace Elem Res 2002; 89: 1-11). Also, it is reported that a significant change (either increase or decrease) from normal tissue distribution of Zn(II) level occurs in patients with various types of cancers and a low serum Zn(II) level is found in patients with colon, bronchogenic and gastrointestinal cancers (Christudoss P, Selvakumar R, Pulimood A B, et al. Tissue zinc levels in precancerous tissue in the gastrointestinal tract of azoxymethane (AOM)-treated rats. Exp Toxicol Pathol 2008; 59: 313-8).

The mechanism by which the serum and tissue Zn(II) levels decrease in various cancers is still unclear and it is not yet certain whether the changed Zn(II) levels have any relationship with carcinogenesis. However, some researches suggest that the Cu(I)/Zn(II) ratio is a good index of the extent and prognosis of gastrointestinal cancer (Gupta S K, Singh S P, Shukla V K. Copper, zinc, and Cu/Zn ratio in carcinoma of the gallbladder. J Surg Oncol 2005; 91: 204-8). However, these researches are mostly based on complicated procedures such as atomic absorption spectroscopy, which require a tissue sample to be analyzed be ashed and dried at 500° C.

An attractive approach to determination of Cu(I)/Zn(II) level as well as 3-dimensional (3-D) distribution of the Cu(I)/Zn(II) ratio in colon tissue is to employ multiphoton microscopy (MPM). MPM, wherein two or more near-infrared photons are used as excitation source, is receiving a lot of attentions in biological and medical fields because of its distinct advantages (Zipfel W R, Williams R M, Webb W W. Nonlinear magic: multiphoton microscopy in the biosciences. Nat Biotechnol 2003; 21: 1369-77). The advantages include higher penetration depth (˜500 μm), reduced photodamage, ability of imaging obscure sample and ignorable background autofluorescence, as compared to OPM. As a result, tissue can be imaged without damage for a longer time (˜1 hr), with higher resolution (<300 nm). Besides, hundreds of sectional images can be obtained along the z-direction at 300 nm intervals without having to slice the tissue (Rogart J N, Nagata J, Loeser C S, et al. Multiphoton imaging can be used for microscopic examination of intact human gastrointestinal mucosa ex vivo. Clin Gastroenterol Hepatol 2008; 6: 95-101). From the obtained images, the 3-D distribution of metal ions can be visualized. However, nothing has been reported about Cu(I)/Zn(II) level in colon tissue and 3-D distribution of the Cu(I)/Zn(II) ratio.

### SUMMARY

The present disclosure is directed to providing a copper(I) ion-selective fluorescent probe capable of detecting free copper(I) ions inside cells for a long time with high selectivity and sensitivity for copper(I) ion, with a penetration depth longer than 90 μm in living cells and tissues and without the problems of mistargeting and photobleaching.

The present disclosure is also directed to providing a method for preparing the fluorescent probe.

The present disclosure is also directed to providing a method for diagnosing malignant disease using the fluorescent probe.

The present disclosure is also directed to providing a kit for diagnosing malignant disease using the fluorescent probe.

In one general aspect, the present disclosure provides a copper(I) ion-selective fluorescent probe of Chemical Formula 1:

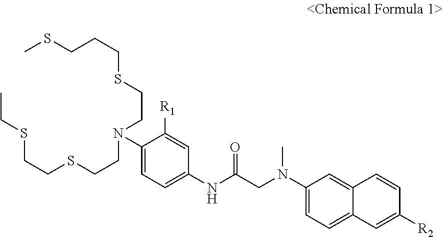

wherein R₁ is hydrogen, C₁-C₁₀ alkyl or C₁-C₁₀ alkoxy, R₂ is —COCH₃,

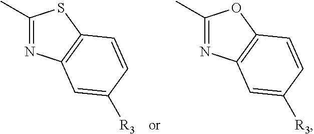

and R₃ is hydrogen or C₁-C₁₀ alkoxy.

In an exemplary embodiment of the present disclosure, R₁ is hydrogen, methyl or methoxy.

In another exemplary embodiment of the present disclosure, R₃ is hydrogen or methoxy.

In another exemplary embodiment of the present disclosure, the compound of Chemical Formula 1 is a compound of Chemical Formula 2:

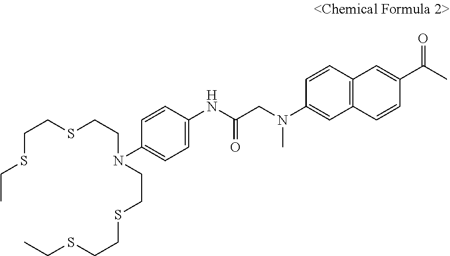 [SMILES: CCSCCSCCN(CCSCCSCC)c1ccc(NC(=O)CN(C)c2ccc3cc(C(C)=O)ccc3c2)cc1]

In another general aspect, the present disclosure provides a method for preparing a copper(I) ion-selective fluorescent probe, including reacting a compound of Chemical Formula 3 with a mixture of 6-acetyl-2-[N-methyl-N-(carboxymethyl)amino]naphthalene, 1-hydroxybenzotriazole and N,N′-dicyclohexylcarbodiimide to prepare a compound of Chemical Formula 2:

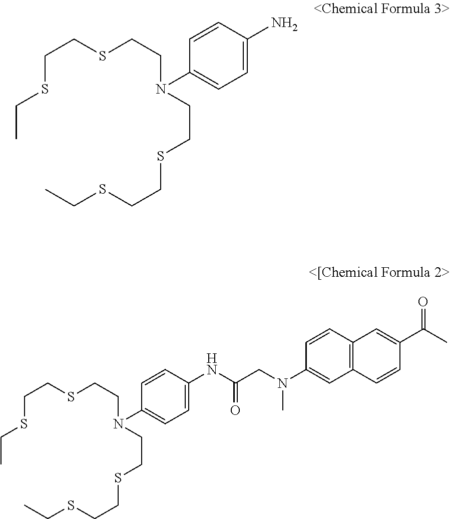 [SMILES: CCSCCSCCN(CCSCCSCC)c1ccc(N)cc1 ; CCSCCSCCN(CCSCCSCC)c1ccc(NC(=O)CN(C)c2ccc3cc(C(C)=O)ccc3c2)cc1]

In an exemplary embodiment of the present disclosure, the compound of Chemical Formula 3 may be prepared by refluxing a mixture of a compound of Chemical Formula 4 and SnCl₂ in an organic solvent:

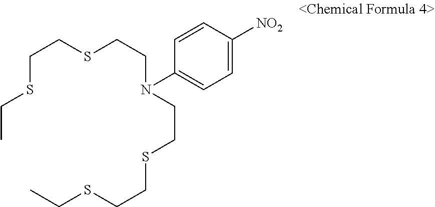 [SMILES: CCSCCSCCN(CCSCCSCC)c1ccc([N+](=O)[O-])cc1]

In another exemplary embodiment of the present disclosure, the compound of Chemical Formula 4 may be prepared by reacting a mixture of a compound of Chemical Formula 5, 2-(ethylthio)ethanethiol and Cs₂CO₃:

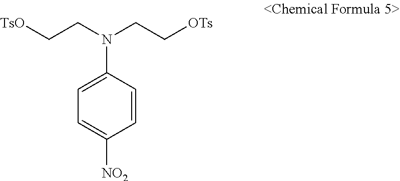

wherein Ts is tosyl.

In another general aspect, the present disclosure provides a method for diagnosing malignant disease, including: labeling copper(I) ions and zinc(II) ions in a biological sample respectively with a copper(I) ion-selective fluorescent probe of Chemical Formula 1 and a zinc(II) ion-selective fluorescent probe; measuring multiphoton fluorescence intensity for the copper(I) ions and zinc(II) ions by multiphoton microscopy; calculating the ratio of the multiphoton fluorescence intensity for the copper(I) ions to the multiphoton fluorescence intensity for the zinc(II) ions from the measured values; and diagnosing malignant disease using the ratio.

In an exemplary embodiment of the present disclosure, the zinc(II) ion-selective fluorescent probe may be a compound of Chemical Formula 6:

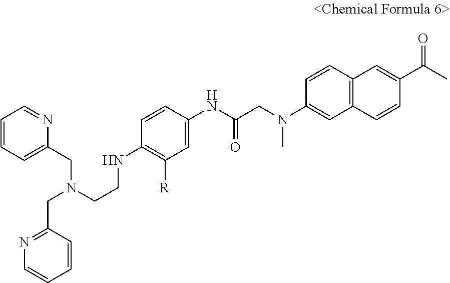

wherein R is hydrogen or OCH₃.

In another exemplary embodiment of the present disclosure, the malignant disease may be respiratory cancer, gastrointestinal cancer or breast cancer.

In another exemplary embodiment of the present disclosure, the malignant disease may be diagnosed when the ratio is between 1.657 and 2.169.

In another general aspect, the present disclosure provides a kit for diagnosing malignant disease including: a first probe-attached portion to which a copper(I) ion-selective fluorescent probe of Chemical Formula 1 is attached; a second probe-attached portion to which a zinc(II) ion-selective fluorescent probe is attached; and a biological sample introducing unit introducing a biological sample to the first and second probe-attached portions.

In an exemplary embodiment of the present disclosure, the zinc(II) ion-selective fluorescent probe may be a compound of Chemical Formula 6:

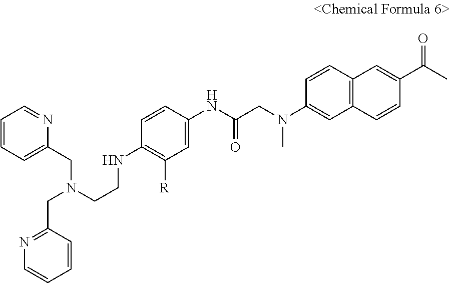

wherein R is hydrogen or OCH₃.

### BRIEF DESCRIPTION OF THE DRAWINGS

The above and other objects, features and advantages of the present disclosure will become apparent from the following description of certain exemplary embodiments given in conjunction with the accompanying drawings, in which:

FIGS. 1A and 1B show normalized absorption (FIG. 1A) and fluorescence (FIG. 1B) spectra of a compound of Chemical Formula 2 in 1,4-dioxane, DMF, ethanol and HEPES buffer ([HEPES]=20 mM, pH 7.0);

FIGS. 2A and 2B show one-photon absorption (FIG. 2A) and fluorescence (FIG. 2B) spectra obtained after adding copper(I) ions to a compound of Chemical Formula 2 in HEPES buffer;

FIG. 3 shows two-photon fluorescence spectra obtained after adding copper(I) ions to a compound of Chemical Formula 2 in HEPES buffer;

FIG. 4 shows a one-photon fluorescence titration curve (open circles) and a two-photon fluorescence titration curve for complexation between a compound of Chemical Formula 2 and free copper(I) ions (0-360 pM);

FIG. 5 shows reactivity of a compound of Chemical Formula 2 for copper(I) ions as compared to competing metal ions as relative fluorescence intensity;

FIG. 6 shows one-photon fluorescence intensity of a compound of Chemical Formula 2 (2 μM) in HEPES buffer in the presence of 0 μM (open circles) and 2 μM (filled circles) copper(I) ions measured at different pH's;

FIG. 7 shows two-photon fluorescence spectra of a compound of Chemical Formula 2 and BODIPY as reference compound in the presence of copper(I) ions;

FIG. 8 shows a two-photon microscopic image of HeLa cells cultured at 37° C. for 20 minutes with a compound of Chemical Formula 2 (2 μM);

FIG. 9 shows a bright-field image of a hippocampal slice of a 2-day-old mouse after culturing with a compound of Chemical Formula 2;

FIG. 10 shows an image obtained by combining 15 two-photon microscopic images of hippocampal slices of a 2-day-old mouse after culturing with a compound of Chemical Formula 2 at varying depths;

FIG. 11 schematically shows a kit according to the present disclosure;

FIG. 12 shows multiphoton microscopic images of HCT 116 and HT-29 colon cancer cells and normal NIH 3T3 cells obtained after labeling with a copper(I) ion-selective fluorescent probe of Chemical Formula 2 and a zinc(II) ion-selective fluorescent probe of Chemical Formula 6;

FIG. 13 shows multiphoton microscopic images of normal, adenoma and cancer tissues obtained along the z-direction over the depths of 100-220 μm after labeling with a copper(I) ion-selective fluorescent probe of Chemical Formula 2;

FIG. 14 shows multiphoton microscopic images of normal, adenoma and cancer tissues obtained along the z-direction over the depths of 100-220 μm after labeling with a zinc(II) ion-selective fluorescent probe of Chemical Formula 6; and

FIG. 15 shows colonoscopic images (left bottom), bright-field images and multiphoton microscopic images of normal, adenoma and cancer tissues labeled with a compound of Chemical Formula 2 (20 μM, middle bottom) and a compound of Chemical Formula 6 (20 μM, right bottom) obtained at a depth of 120 μm, and relative multiphoton-excited fluorescence (MPEF) intensity of tissues labeled with a compound of Chemical Formula 2 (20 μM, left top) and a compound of Chemical Formula 6 (20 μM, right top).

### DETAILED DESCRIPTION OF EMBODIMENTS

Hereinafter, exemplary embodiments will be described in detail with reference to the accompanying drawings.

The present disclosure provides a copper(I) ion-selective fluorescent probe of Chemical Formula 1:

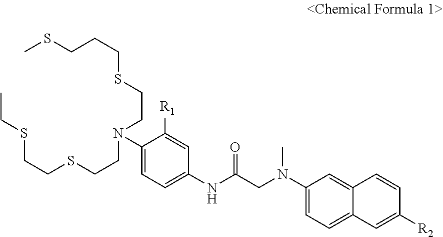

wherein R₁ is hydrogen, C₁-C₁₀ alkyl or C₁-C₁₀ alkoxy, R₂ is —COCH₃,

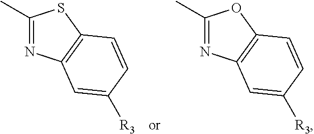

and R₃ is hydrogen or C₁-C₁₀ alkoxy.

In Chemical Formula 1, R₁ may be hydrogen, methyl or methoxy, and R₃ may be hydrogen or methoxy. And, the compound of Chemical Formula 1 may be a compound of Chemical Formula 2:

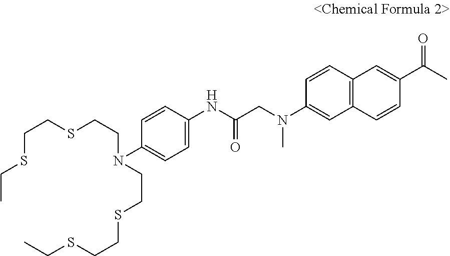 [SMILES: CCSCCSCCN(CCSCCSCC)c1ccc(NC(=O)CN(C)c2ccc3cc(C(C)=O)ccc3c2)cc1]

The fluorescent probe according to the present disclosure has very high selectivity for copper(I) ions in cells. As demonstrated in the following experimental examples of comparing with various competing metal ions such as Na⁺, K⁺, Mg²⁺, Ca²⁺, Mn²⁺, Fe²⁺, Co²⁺, Ni²⁺, Cu²⁺ and Zn²⁺, the fluorescent probe according to the present disclosure reacts selectively with copper(I) ion with very high reactivity in vivo. Moreover, the reaction signal from the fluorescent probe according to the present disclosure has been found to be irrelevant of the pH of reaction solutions within the biologically significant pH ranges, suggesting that the probe according to the present disclosure is capable of detecting copper(I) ions without interference by pH.

Also, the probe according to the present disclosure exhibits remarkably higher Fd value when compared with existing dyes such as BODIPY. This means that a sample stained with the probe according to the present disclosure gives a much brighter TPM image than one stained with the existing dye.

In addition, in order to verify if the probe according to the present disclosure is suitable for in vivo imaging, detection of copper(I) ions was conducted in cultured HeLa cells. TPM images were obtained for mouse hippocampal tissue slices in order to verify if copper(I) ions present deep in the living tissue can be detected. As a result, the probe according to the present disclosure has been confirmed to be applicable for detection of copper(I) ions in vivo, with excellent detection sensitivity at varying tissue depths.

The present disclosure also provides a method for preparing a copper(I) ion-selective fluorescent probe, comprising reacting a compound of Chemical Formula 3 with a mixture of 6-acetyl-2-[N-methyl-N-(carboxymethyl)amino]naphthalene, 1-hydroxybenzotriazole and N,N′-dicyclohexylcarbodiimide to prepare a compound of Chemical Formula 2:

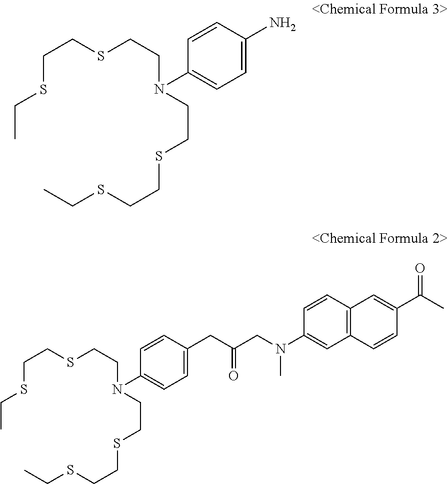 [SMILES: CCSCCSCCN(CCSCCSCC)c1ccc(N)cc1 ; CCSCCSCCN(CCSCCSCC)c1ccc(CC(=O)CN(C)c2ccc3cc(C(C)=O)ccc3c2)cc1]

The reaction may be performed by stirring a mixture of 6-acetyl-2-[N-methyl-N-(carboxymethyl)amino]naphthalene, 1-hydroxybenzotriazole and N,N′-dicyclohexylcarbodiimide in an organic solvent such as CH₂Cl₂, adding the compound of Chemical Formula 3 to the mixture solution, and then further stirring the mixture. The 6-acetyl-2-[N-methyl-N-(carboxymethyl)amino]naphthalene may be synthesized according to a known method (H. M. Kim, C. Jung, B. R. Kim, S. Y. Jung, J. H. Hong, Y. G. Ko, K. J. Lee, B. R. Cho, Angew. Chem. Int. Ed. 2007, 46, 3460).

Specifically, the compound of Chemical Formula 3 may be prepared by refluxing a mixture of a compound of Chemical Formula 4 and SnCl₂ in an organic solvent:

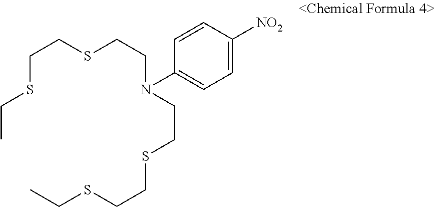 [SMILES: CCSCCSCCN(CCSCCSCC)c1ccc([N+](=O)[O-])cc1]

The organic solvent may be, for example, a mixture of THF and ethanol. The compound of Chemical Formula 3 may be obtained by removing the solvent in vacuum, treating with an alkaline solution such as NaOH, and then extracting and drying.

Specifically, the compound of Chemical Formula 4 may be prepared by reacting a mixture of a compound of Chemical Formula 5, 2-(ethylthio)ethanethiol and Cs₂CO₃:

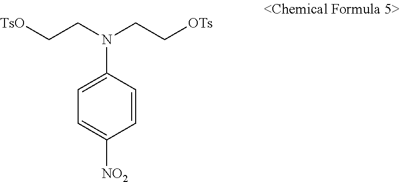

wherein Ts is tosyl.

For example, the compound of Chemical Formula 4 may be obtained by dissolving a mixture of 2-(ethylthio)ethanethiol and Cs₂CO₃ in DMF, slowly adding the compound of Chemical Formula 5 solution to the DMF solvent and stirring, evaporating the solvent, and then extracting and drying. The compound of Chemical Formula 5 may be synthesized according to a known method (M. W. Glenny, L. G. A. van de Water, J. M. Vere, A. J. Blake, C. Wilson, W. L. Driessen, J. Reedijk, M. Schroder, Polyhedron. 2006, 25, 599).

An exemplary reaction scheme for preparing the compound of Chemical Formula 2 from the compound of Chemical Formula 5 is shown in Scheme 1.

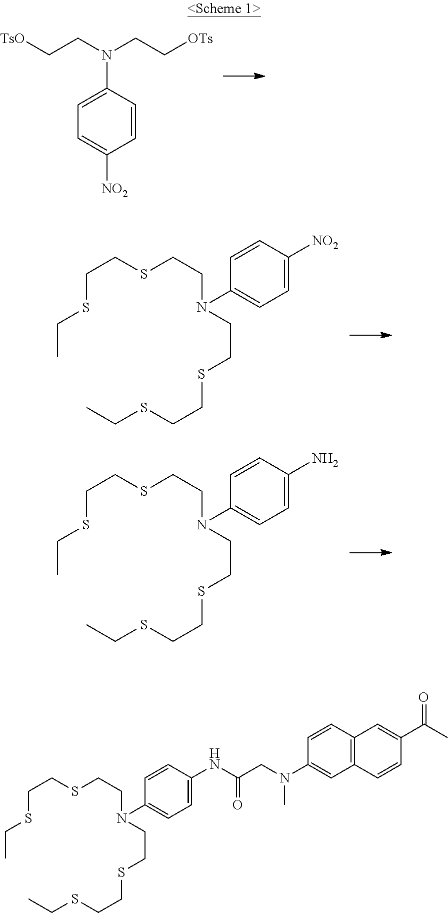

wherein Ts is tosyl.

In accordance with the present disclosure, the compound of Chemical Formula 1 may be used to determine Cu(I)/Zn(II) level and 3-dimensional distribution of the Cu(I)/Zn(II) ratio in normal tissues and tissues of malignant disease, thus enabling effective diagnosis of the malignant disease. Multiphoton microscopy, the leading fluorescence microscopic technique used for thick tissues and living animals, is a useful tool for cancer researchers who study angiogenesis and metastasis in vivo, immunologists who investigate lymphocyte transportation, and embryologists who investigate developing hamster embryos. The inventors of the present disclosure have noted that patients with specific malignant diseases show increased Cu(I)/Zn(II) ratio and have completed the present disclosure.

A method for diagnosing malignant disease according to the present disclosure comprises: labeling copper(I) ions and zinc(II) ions in a biological sample respectively with the copper(I) ion-selective fluorescent probe of Chemical Formula 1 and a zinc(II) ion-selective fluorescent probe; measuring multiphoton fluorescence intensity for the copper(I) ions and zinc(II) ions by multiphoton microscopy; calculating the ratio of the multiphoton fluorescence intensity for the copper(I) ions to the multiphoton fluorescence intensity for the zinc(II) ions from the measured values; and diagnosing malignant disease using the ratio.

As described above, the copper(I) ion-selective fluorescent probe of Chemical Formula 1 has higher selectivity for copper(I) ions as compared to other metal ions. The zinc(II) ion-selective fluorescent probe may be any substance that has high selectivity for zinc(II) ions as compared to other metal ions and has superior two-photon excited fluorescence intensity, without particular limitation. In this regard, the inventors of the present disclosure have disclosed a two-photon dye for detecting free zinc ions in the cytoplasm in Korean Patent Publication No. 2009-0118412 as the zinc(II) ion-selective fluorescent probe. The two-photon dye has high selectivity for Zn²⁺ and enables very effective, long-term monitoring of free Zn²⁺ in the cytoplasm located deep from the surface. A compound of Chemical Formula 6 disclosed in the publication may be used as the zinc(II) ion-selective fluorescent probe:

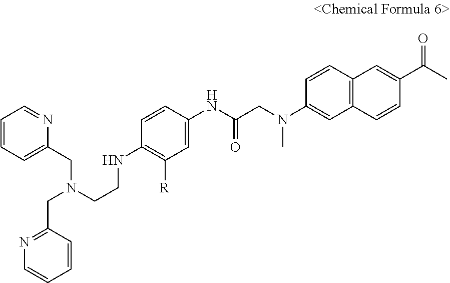

wherein R is hydrogen or OCH₃.

Specific examples of the malignant disease to which the present disclosure is applicable include gastrointestinal cancer such as colon cancer or rectal cancer, respiratory cancer such as bronchogenic cancer or lung cancer, or breast cancer. However, the scope of the present disclosure is not limited to the above diseases but the present disclosure may be applicable to any disease associated with change in the Cu(I)/Zn(II) ratio in vivo.

As seen from Table 2 given in the Example section, the Cu(I)/Zn(II) ratio used in the present disclosure as an index for diagnosing malignant disease is about between 0.398 and 0.704 in normal cells or tissues but is between 1.657 and 2.169 in cells or tissues with malignant disease. Accordingly, when the ratio is in the range, it may be determined that the sample has malignant disease.

The present disclosure also provides a kit for diagnosing malignant disease using the copper(I) ion-selective fluorescent probe. The kit according to the present disclosure comprises: a first probe-attached portion to which the copper(I) ion-selective fluorescent probe of Chemical Formula 1 is attached; a second probe-attached portion to which a zinc(II) ion-selective fluorescent probe is attached; and a biological sample introducing unit introducing a biological sample to the first and second probe-attached portions. As described above, the zinc(II) ion-selective fluorescent probe may be the compound of Chemical Formula 6.

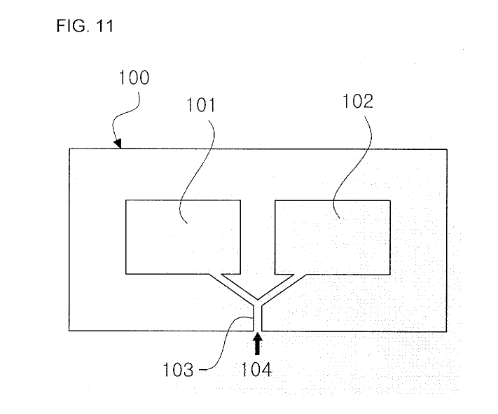

FIG. 11 schematically shows a kit according to the present disclosure. Referring to FIG. 11, the outer surface of a kit 100 may be made of glass or transparent plastic for easy fluorescence analysis. The kit 100 has a first probe-attached portion 101 for analysis of copper(I) ions, a second probe-attached portion 102 for analysis of zinc(II) ions, and a biological sample introducing unit 103 for introducing a biological sample 104 into the kit. As described above, the first and second probes may be the compounds of Chemical Formulae 1 and 6, respectively, and the malignant disease to be diagnosed may be respiratory cancer, gastrointestinal cancer or breast cancer. Thus, after introducing a biological sample into the kit and recording multiphoton fluorescence intensity measured by the first probe-attached portion 101 and the second probe-attached portion 102 by multiphoton microscopy, the ratio of the fluorescence intensity measured by the first probe-attached portion to the fluorescence intensity measured by the second probe-attached portion, i.e. the ratio of the fluorescence intensity measured by the copper(I) ion-selective probe to the fluorescence intensity measured by the zinc(II) ion-selective probe may be calculated to diagnose malignant disease.

### EXAMPLES

The examples and experiments will now be described. The following examples and experiments are for illustrative purposes only and not intended to limit the scope of this disclosure.

### Preparation Examples

### Preparation of Compound of Chemical Formula 2

A compound of Chemical Formula 2 which is the fluorescent probe according to the present disclosure was prepared as follows:

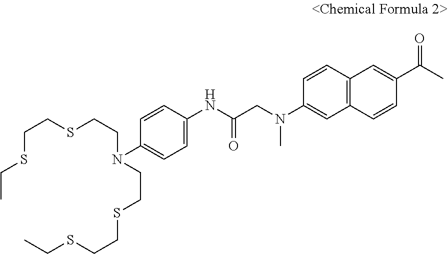 [SMILES: CCSCCSCCN(CCSCCSCC)c1ccc(NC(=O)CN(C)c2ccc3cc(C(C)=O)ccc3c2)cc1]

### Preparation Example 1.1

### Preparation of N,N-bis(tosylethyl)-4-nitroaniline (Chemical Formula 5)

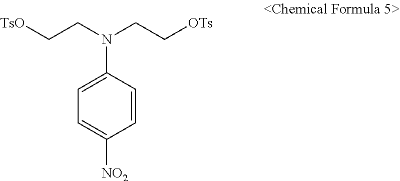 [SMILES: Cc1ccc(S(=O)(=O)OCCN(CCOS(=O)(=O)c2ccc(C)cc2)c2ccc([N+](=O)[O-])cc2)cc1]

In the above chemical formula, Ts is tosyl.

The compound of Chemical Formula 5 was prepared according to a method described in the literature (M. W. Glenny, L. G. A. van de Water, J. M. Vere, A. J. Blake, C. Wilson, W. L. Driessen, J. Reedijk, M. Schroder, Polyhedron. 2006, 25, 599).

### Preparation Example 1.2

### Preparation of N,N-bis{2-[2-(ethylthio)ethylthio]ethyl}-4-nitroaniline (Chemical Formula 4)

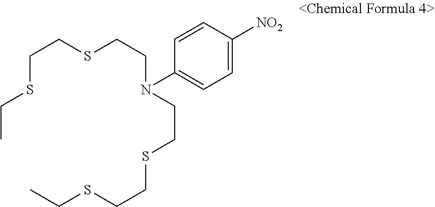 [SMILES: CCSCCSCCN(CCSCCSCC)c1ccc([N+](=O)[O-])cc1]

A mixture solution (50 mL) of 2-(ethylthio)ethanethiol (0.60 g, 4.9 mmol) and Cs₂CO₃ (1.9 g, 5.8 mmol) in DMF was slowly added to a solution (50 mL) of the compound of Chemical Formula 5 (1.2 g, 2.3 mmol) in DMF at 160° C., and the mixture was stirred for 24 hours. After evaporating the solvent, the residue was poured into water (100 mL) and then stirred for 1 hour. The resulting product was extracted with CH₂Cl₂, dried with MgSO₄, filtered and evaporated. The product was purified by flash column chromatography using n-hexane/ethyl acetate (3:1 to 1:1) as eluent. Then, after concentration under reduced pressure, pale yellow oil was obtained.

Yield: 0.77 g (63%); IR (KBr): 1594 cm⁻¹; ¹H NMR (300 MHz, CDCl₃): δ 8.14 (2H, d, J=7.1 Hz), 6.63 (2H, d, J=7.1 Hz), 3.67 (4H, t, J=5.4 Hz), 2.85 (4H, q, J=5.6 Hz), 2.82-2.73 (12H, m), 1.27 (6H, t, J=5.6 Hz); ¹³C NMR (100 MHz, CDCl₃): δ=151.7, 137.9, 126.7, 110.6, 51.8, 32.9, 32.1, 29.6, 26.4, 15.0 ppm; HRMS (FAB⁺): m/z calculated for [C₁₈H₃₀N₂O₂S₄+H⁺]: 435.1267, measured: 435.1269.

### Preparation Example 1.3

### Preparation of N,N-bis{2-[2-(ethylthio)ethylthio]ethyl}benzene-1,4-diamine (Chemical Formula 3)

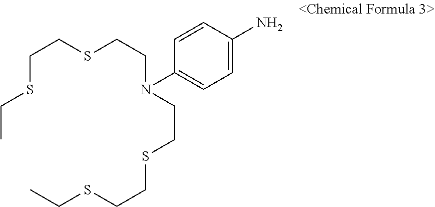 [SMILES: CCSCCSCCN(CCSCCSCC)c1ccc(N)cc1]

A mixture of the compound of Chemical Formula 4 (0.30 g, 0.69 mmol) prepared in Preparation Example 1.2 and SnCl₂ (1.5 g, 6.9 mmol) was refluxed for 12 hours in a THF/ethanol solvent (1/1, 80 mL). After removing the solvent in vacuum, the crude product was treated with an aqueous NaOH solution. When the solution turned alkaline, the product was extracted with CH₂Cl₂ and dried with MgSO₄. Then, after removing the solvent in vacuum, dark brown oil was obtained.

Yield: 0.16 g (56%); (56%); IR (KBr): 3480, 3340 cm⁻¹; ¹H NMR (300 MHz, CDCl₃): δ 6.67 (4H, m), 3.41 (4H, t, J=7.6 Hz), 2.73 (12H, m), 2.56 (4H, q, J=7.4 Hz), 1.26 (6H, t, J=7.4); ¹³C NMR (100 MHz, CDCl₃): δ=135.9, 133.6, 115.1, 112.6, 51.9, 32.7, 32.0, 29.6, 26.3, 15.0 ppm; HRMS (FAB⁺): m/z calculated for [C₁₈H₃₂N₂S₄+H⁺]: 404.1448, measured: 404.1451.

### Preparation Example 1.4

### Preparation of Compound of Chemical Formula 2

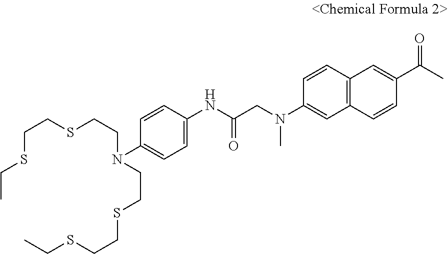 [SMILES: CCSCCSCCN(CCSCCSCC)c1ccc(NC(=O)CN(C)c2ccc3cc(C(C)=O)ccc3c2)cc1]

A mixture of 6-acetyl-2-[N-methyl-N-(carboxymethyl)amino]naphthalene (0.13 g, 0.50 mmol), 1-hydroxybenzotriazole (0.07 g, 0.50 mmol), and N,N′-dicyclohexylcarbodiimide (0.10 g, 0.50 mmol) in CH₂Cl₂ (30 mL) was stirred for 1 hour. After adding the compound of Chemical Formula 3 (0.16 g, 0.42 mmol) in CH₂Cl₂ (15 mL), the mixture was stirred for 4 hours under N₂ atmosphere. The resulting mixture was filtered and the filtrate was extracted with CH₂Cl₂, washed with saturated NaHCO₃ (aq), dried with Na₂SO₄, filtered and evaporated. The crude product was extracted with CH₂Cl₂, dried with MgSO₄, filtered and evaporated. The product was purified by column chromatography using hexane/ethyl acetate/CHCl₃ (1:1:1) as eluent.

Yield 0.14 g (50%); melting point 150.3° C.; IR (KBr): 3450, 1650, 1616 cm⁻¹; ¹H NMR (400 MHz, CDCl₃): 8.37 (1H, d, J=1.2 Hz), 8.05 (1H, br s), 7.99 (1H, dd, J=1.2, 6.9 Hz), 7.89 (1H, d, J=6.9 Hz), 7.72 (1H, d, J=6.9 Hz), 7.34 (2H, d, J=6.8 Hz), 7.19 (1H, dd, J=1.8, 6.9 Hz), 7.07 (1H, d, J=1.8 Hz), 6.61 (2H, d, J=6.8 Hz), 4.12 (2H, s), 3.52 (4H, t, J=5.5 Hz), 3.25 (3H, s), 2.77-2.71 (12H, m), 2.69 (3H, s), 2.56 (4H, q, J=5.6 Hz), 1.25 (6H, t, 5.6 Hz); ¹³C NMR (400 MHz, CDCl₃): δ=198.0, 167.9, 149.2, 137.3, 132.3, 131.6, 130.4, 127.0, 126.6, 125.2, 122.6, 116.6, 112.5, 107.6, 59.4, 51.9, 40.4, 32.7, 32.6, 31.9, 31.8, 29.6, 26.8, 26.3, 15.0 ppm; HRMS (FAB⁺): m/z calculated for [C₃₃H₄₅N₃O₂S₄+H⁺]: 644.2473, measured: 644.2474.

### Example 1

### Measurement of Absorption and Fluorescence Spectra

Absorption spectra were obtained using a Hewlett-Packard 8453 diode array spectrophotometer and fluorescence spectra were obtained using an Amico-Bowman series 2 luminescence spectrometer equipped with a 1-cm standard quartz cell. Fluorescence quantum yield was determined according to the literature using Coumarin 307 as reference compound (J. N. Demas, G. A. Crosby, J. Phys. Chem. 1971, 75, 991).

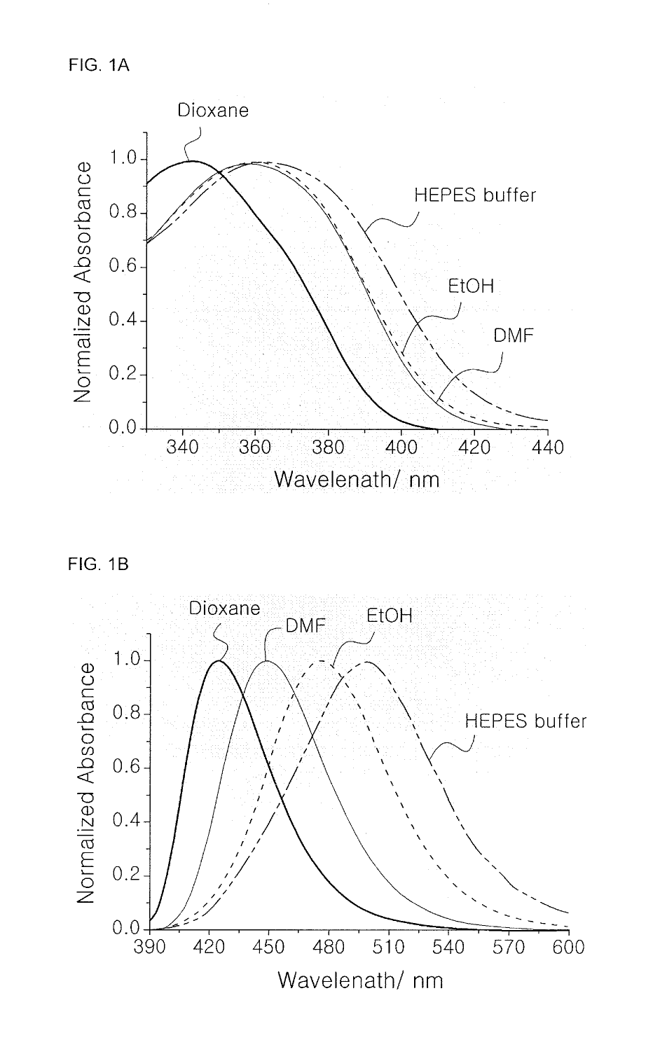

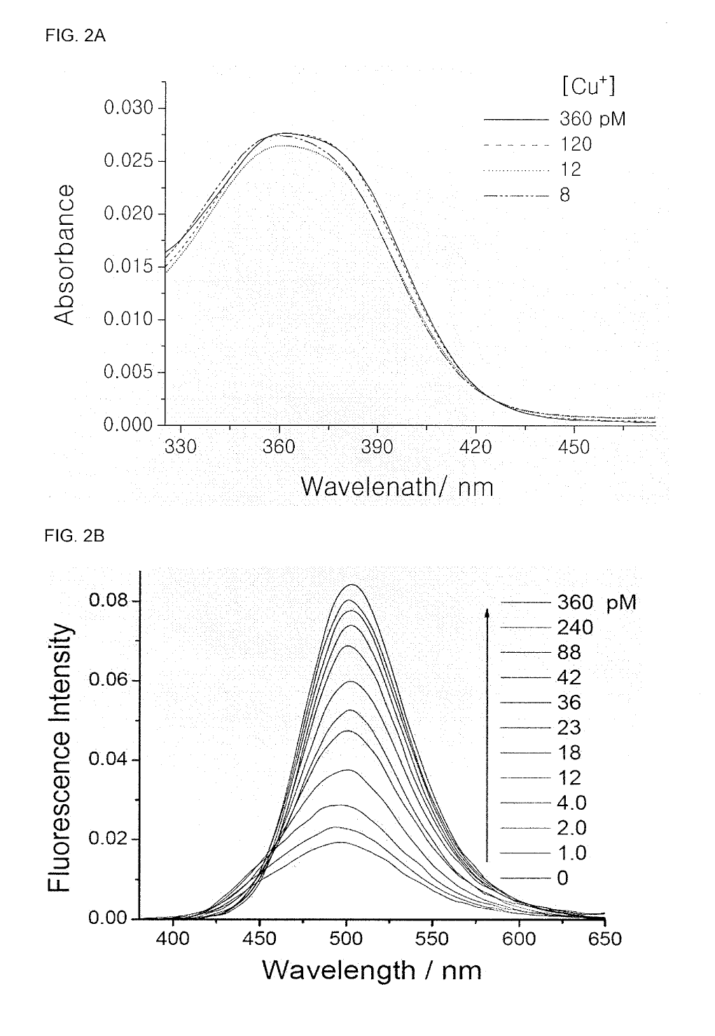

FIGS. 1A and 1B show normalized absorption (FIG. 1A) and fluorescence (FIG. 1B) spectra of the compound of Chemical Formula 2 in 1,4-dioxane, DMF, ethanol and HEPES buffer ([HEPES]=20 mM, pH 7.0). Light with a wavelength of 365 nm was used for excitation. Solubility of the compound of Chemical Formula 2 in HEPES buffer ([HEPES]=20 mM, pH 7.0) was measured as 4.0 μM by the fluorescence method disclosed in the literature (H. M. Kim and B. R. Cho, Acc. Chem. Res., 2009, 42, 863; M. K. Kim, C. S. Lim, J. T. Hong, J. H. Han, H. Y. Jang, H. M. Kim and B. R. Cho, Angew. Chem., Int. Ed., 2010, 49, 364; J. H. Lee, C. S. Lim, Y. S. Tian, J. H. Han and B. R. Cho, J. Am. Chem. Soc., 2010, 132, 1216; H. M. Kim and B. R. Cho, Chem. Asian J., 2011, 6, 58), which is sufficient for staining of cells.

Referring to FIGS. 1A and 1B, maximum absorption and fluorescence of the compound of Chemical Formula 2 showed significant red shift depending on solvent polarity (1,4-dioxane<ethanol<HEPES buffer). The shift of maximum peaks for the short-wavelength light was higher for the fluorescence spectra (65 nm) than for the absorption spectrum (17 nm). This reflects that the compound of Chemical Formula 2 is a useful polar dye.

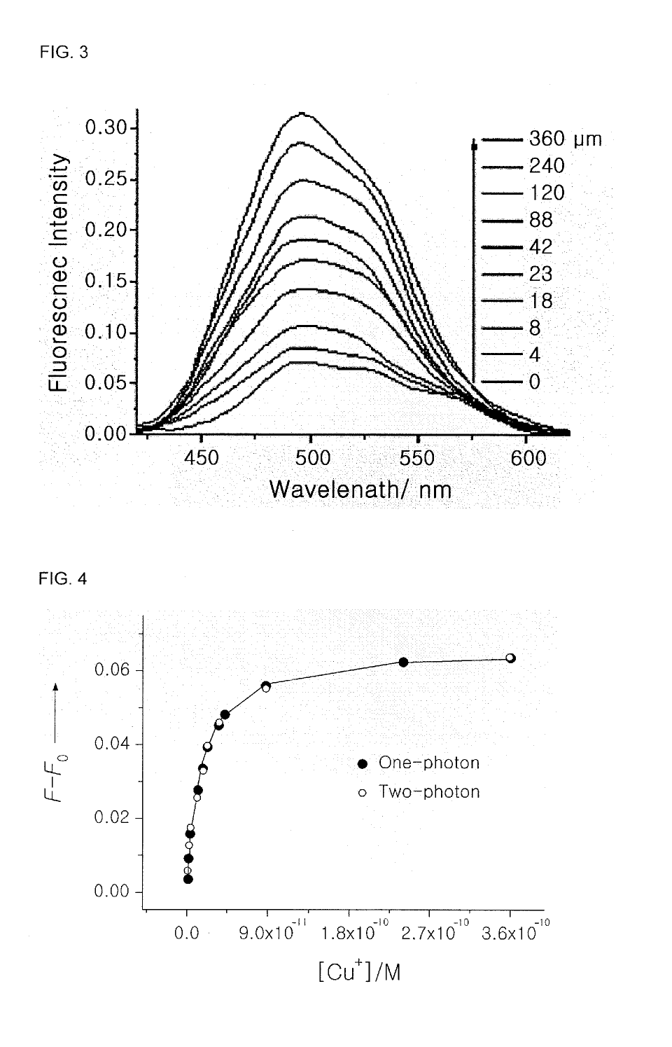

FIGS. 2A and 2B show one-photon absorption (FIG. 2A) and fluorescence (FIG. 2B) spectra for the compound of Chemical Formula 2 (2 μM) in the presence of free copper(I) ions (0-360 pM). Referring to FIGS. 2A and 2B, when copper(I) ions were added to the compound of Chemical Formula 2 in HEPES buffer, the fluorescence intensity increased gradually with the concentration of the copper(I) ions, without effect on the absorption spectrum. It may be because the metal ion forms a complex with the compound of Chemical Formula 2 and blocks photoinduced electron transfer (PeT). When the concentration of free copper(I) ion was 360 pM, the fluorescence intensity increased by about 4 times. A similar behavior was also observed for two-photon fluorescence spectra (see FIG. 3).

Dissociation constants (K_{d}) of the compound of Chemical Formula 2 for one-photon and two-photon processes were calculated from fluorescence titration curves (see FIG. 4) (HEPES buffer was used. [HEPES]=20 mM, [thiourea]=100 μM, pH=7.0). The fluorescence titration curves were consistent with the 1:1 binding model, suggesting that the compound of Chemical Formula 2 and the copper(I) ion forms a 1:1 complex. The dissociation constants for the copper(I) ion were respectively 16±2 pM and 20±3 pM for the one-photon and two-photon processes, which indicates that the compound of Chemical Formula 2 can be usefully used for detection of copper(I) ions in pM level.

### Example 2

### Reactivity of Competing Ions

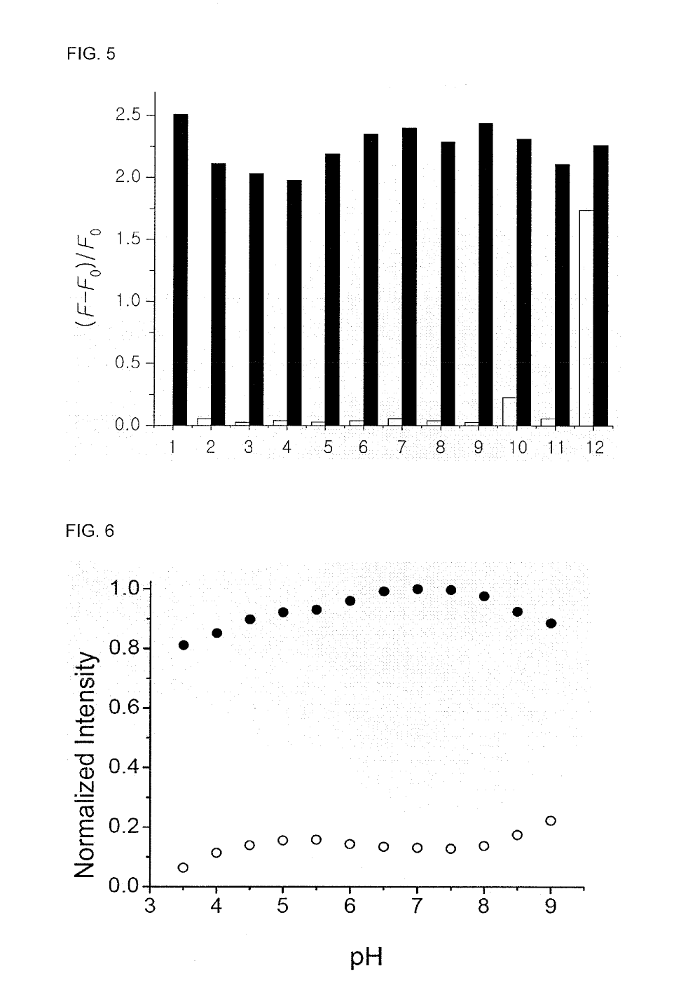

FIG. 5 shows reactivity of the compound of Chemical Formula 2 for copper(I) ions as compared to competing metal ions as relative fluorescence intensity. In FIG. 5, open bars show relative fluorescence intensity of the compound of Chemical Formula 2 (2 μM) resulting from reaction with Na⁺, K⁺, Mg²⁺ and Ca²⁺ (1 mM), Hg²⁺ (2 μM) and other cations (50 mM) in 20 mM HEPES buffer (pH 7.0) before adding copper(I) ions, and filled bars show relative fluorescence intensity after adding 2 μM copper(I) ions (The cations indicated as numbers below the bars are as follows: (1) Cu⁺; (2) Na⁺; (3) K⁺; (4) Mg²⁺; (5) Ca²⁺; (6) Mn²⁺; (7) Fe²⁺, (8) Co²⁺; (9) Ni²⁺; (10) Cu²⁺; (11) Zn²⁺; (12) Hg²⁺). Referring to FIG. 5, it can be seen that the compound of Chemical Formula 2 is not interfered with by the presence of alkali metal or alkaline earth metal ions such as Na⁺, Ka⁺, Mg²⁺ and Ca²⁺ and transition metal ions such as Mn²⁺, Fe²⁺, Co²⁺, Ni²⁺, Cu²⁺ and Zn²⁺. Although the only exception is Hg²⁺, considering that the concentration of Hg²″ in living organism is ignorable, it can be concluded that the compound of Chemical Formula 2 is capable of selectively detecting copper(I) ions in cells.

FIG. 6 shows one-photon fluorescence intensity of the compound of Chemical Formula 2 (2 μM) in HEPES buffer in the presence of 0 μM (open circles) and 2 μM (filled circles) copper(I) ions measured at different pH's. Light with a wavelength of 365 nm was used for excitation. Referring to FIG. 6, it can be seen that the reactivity of the compound of Chemical Formula 2 is irrelevant of the pH of reaction solutions within the biologically significant pH ranges. Thus, from FIG. 5 and FIG. 6, it can be seen that the compound of Chemical Formula 2 according to the present disclosure is capable of detecting copper(I) ions with little interference by pH or other metal ion.

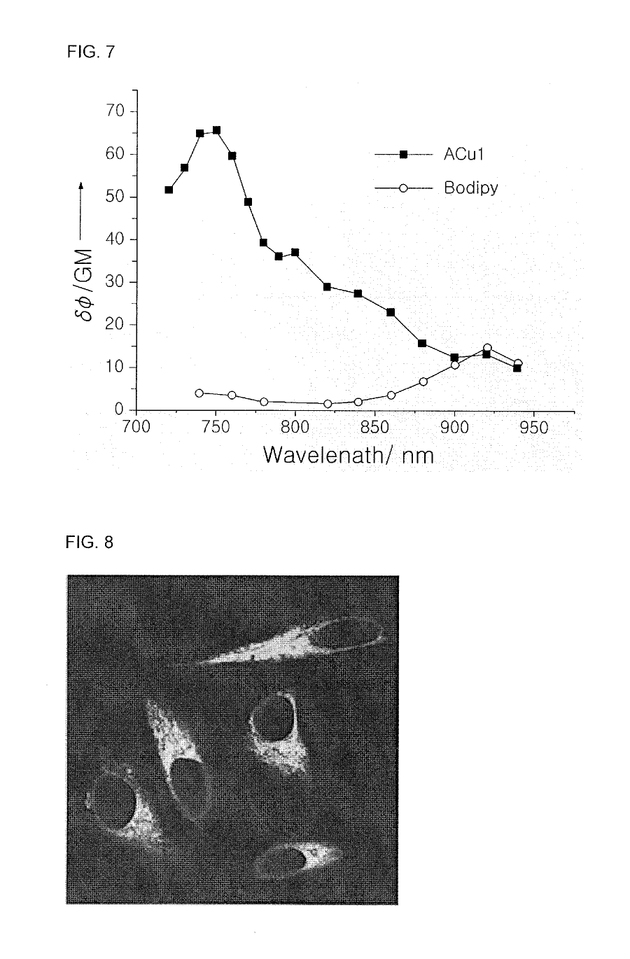

FIG. 7 shows two-photon fluorescence spectra of the compound of Chemical Formula 2 (ACu1) and BODIPY as reference compound in the presence of 2 μM copper(I) ions (The values for BODIPY were cited from C. Xu and W. W. Webb, J. Opt. Soc. Am. B, 1996, 13, 481). Referring to FIG. 7, the two-photon spectrum of the compound of Chemical Formula 2-Cu⁺ complex in HEPES buffer indicated an Fd value of 67 GM at 750 nm, more than 13-fold as compared to BODIPY (<5 GM 750 nm). Accordingly, it can be conjectured that a sample stained with the compound of Chemical Formula 2 gives a much brighter two-photon image than one stained with the BODIPY dye.

### Example 3

### Detection of Copper Ion in Cultured Cells

In order to investigate the applicability of the compound of Chemical Formula 2 in biological cells, copper(I) ions in cultured HeLa cells were detected by two-photon microscopy. FIG. 8 shows a two-photon microscopic image of HeLa cells cultured at 37° C. for 20 minutes with the compound of Chemical Formula 2 (2 μM). Referring to FIG. 8, the HeLa cells labeled with the compound of Chemical Formula 2 give a very bright TPM image, which is thought of as a result of easy loading and very large Fd value of the reaction product.

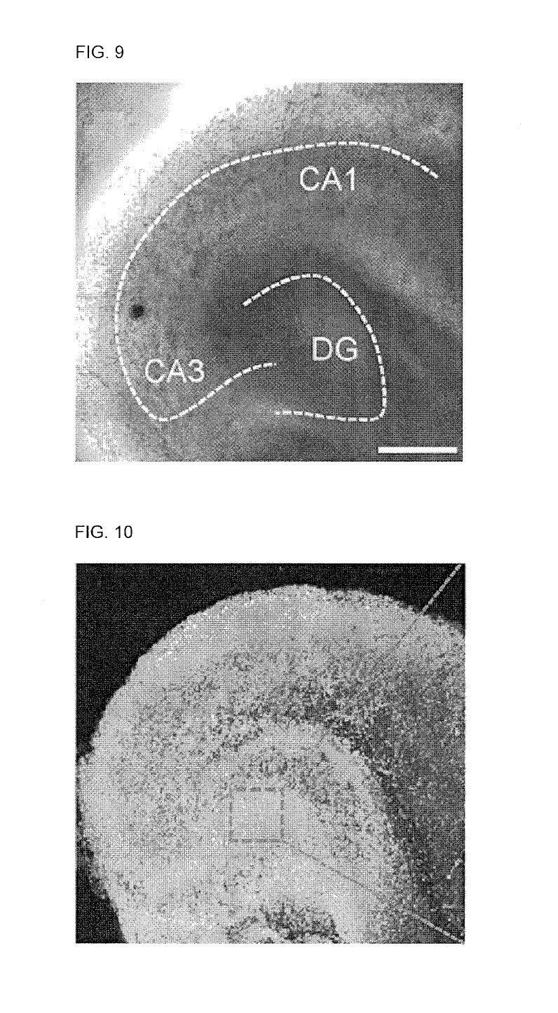

Also, a bright-field image of a hippocampal slice of a 2-day-old mouse was obtained after culturing with the compound of Chemical Formula 2 (20 μM) at 37° C. for 1 hour in order to investigate whether the compound of Chemical Formula 2 is capable of detecting copper(I) ions deep in the living tissue. The result is shown in FIG. 9. Referring to FIG. 9, copper(I) ions were observed not only in the CA1 and CA3 regions but also in the dentate gyrus. Since the brain tissue has a nonhomogeneous structure, 15 TPM images obtained at varying depths of 90-220 μm were combined to visualize the overall distribution of copper(I) ions, which is shown in FIG. 10. Referring to FIG. 10, copper(I) ions were more abundant in the CA1 and CA3 regions than in the dentate gyrus.

### Preparation Example 2

A zinc(II) ion-selective fluorescent probe of Chemical Formula 6 was prepared according to the disclosure of Korean Patent Publication No. 2009-0118412:

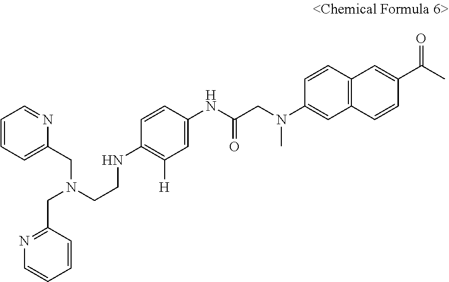 [SMILES: CC(=O)c1ccc2cc(N(C)CC(=O)Nc3ccc(NCCN(Cc4ccccn4)Cc4ccccn4)cc3)ccc2c1]

Dissociation constant (K_{d}^{TP}) of the compound prepared in Preparation Example 2 for the multiphoton process was 1.1 nM. Accordingly, the compound could be used to detect Zn(II) ions in the picomolar (pM) to nanomolar (nM) range by multiphoton microscopy.

### Example 4

### Multiphoton Microscopic Imaging of Colon Cancers

Biological Sample and Multiphoton Microscopic Imaging

HCT 116 and HT-29 colon cancer cells were acquired from the Korean Cell Line Bank. The cells were kept at 37° C. under humidified atmosphere of 5/95 (V/V) CO₂/air. Two days before imaging, the cells were subcultured and seeded on a glass-bottom dish (MatTek). For labeling, growth medium was removed and replaced with fetal bovine serum (FBS)-free RPMI 1640. The cells were cultured at 37° C. for 30 minutes with the compound of Preparation Example 1, the compound of Preparation Example 2 and the synthetic block copolymer Pluronic F-127 (2 μM), and washed 3 times with FBS-free RPMI 1640. The cells were imaged after washing 3 times with phosphate buffered saline (PBS; Gibco). Cu(I)/Zn(II) level was determined by measuring the intensity of multiphoton-excited fluorescence (MPEF).

Discussion

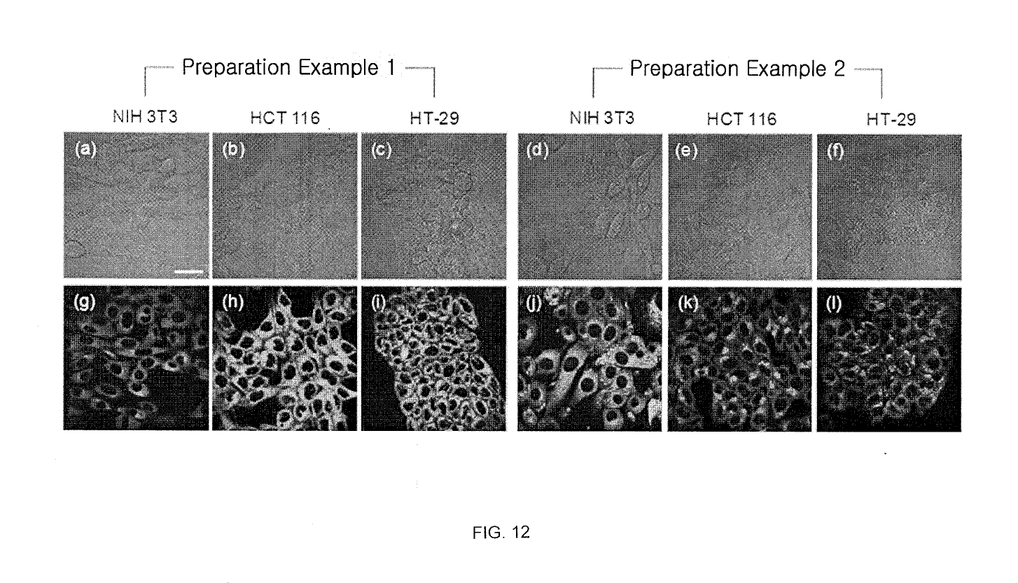

FIG. 12 shows multiphoton microscopic images of HCT 116 and HT-29 colon cancer cells and normal NIH 3T3 cells obtained after labeling with the probes of Preparation Example 1 and Preparation Example 2. The individual images are bright-field images (a-f) and multiphoton microscopic images (g-l) of normal cells (NIH3T3) and colon cancer cells (HCT116, HT-29) labeled with the compound of Preparation Example 1 (g-i) and the compound of Preparation Example 2 (j-l) at 2 μM concentration. TPEF was collected at 500-620 nm after excitation at 750 nm with fs pulse. The scale bars are 300 μm long. The cells shown are representative images from replicate experiments (n=5).

Referring to FIG. 12, the multiphoton microscopic images of the cells labeled with the compounds of Preparation Example 1 and Preparation Example 2 show distribution of Cu(I) and Zn(II) in the cytoplasm. When labeled with the compound of Preparation Example 1, the images of ACT 116 and HT-29 cells were brighter than NIH 3T3 cells. The opposite was observed when the cells were labeled with the compound of Preparation Example 2. Accordingly, it can be seen that Cu(I) is more abundant in colon cancer cells (ACT116 and HT-29) than in normal cells (NIH3T3), whereas Zn(II) is more abundant in normal cells than in colon cancer cells.

### Example 5

### Multiphoton Microscopic Imaging of Colon Tissue

Biological Sample

For selective colonoscopy, outpatients who visited Korea University Anam Hospital were recruited to participate in this study, which was approved by the hospital's Ethics Committee. Written consent was received from the participants. The patients aged 18 years or older who gave written consent were enlisted, and those who have or are suspected of pre-existing bleeding disorder, those whose international normalized ratio exceeds 1.4, those whose number of platelets is below 100,000 or those who took aspirin within 5 days were excluded. During colonoscopic examination, malignant pathological tissues, adenoma tissues and normal mucosal tissues were obtained using biopsy forceps. Standard biopsy forceps (Olympus Medical Systems Corporation, Tokyo, Japan) were used to obtain paired biopsy specimens from the colonic mucosa. Each pair of the biopsy specimens was separated such that one would be imaged for Cu(I) distribution, and the other for Zn(II) distribution. The tissues were put in sterilized bottles containing PBS and stained at 37.8° C. for 1-2 hours with the compound of Preparation Example 1 and the compound of Preparation Example 2 (20 μM) in artificial cerebrospinal fluid.

Multiphoton Fluorescence Microscopic Observation

Multiphoton fluorescence microscopic images were obtained using a DM IRE2 microscope (Leica) equipped with ×100 (NA=1.30 OIL) and ×10 (NA=0.30 DRY) objective lenses by exciting the probes with a mode-locked titanium-sapphire laser source (Coherent Chameleon, 90 MHz, 200 fs) set at wavelength 760 nm and output power 1230 mW, which corresponded to approximately 10 mW average power in the focal plane. To obtain images at 500-620 nm range, internal photomultiplier tubes (PMTs) were used to collect the signals in 8-bit unsigned 512×512 pixels at 400 Hz scan rate.

3-Dimensional Distribution of Cu(I)/Zn(II) and Relative Cu(I)/Zn(II) Level in Colon Tissue

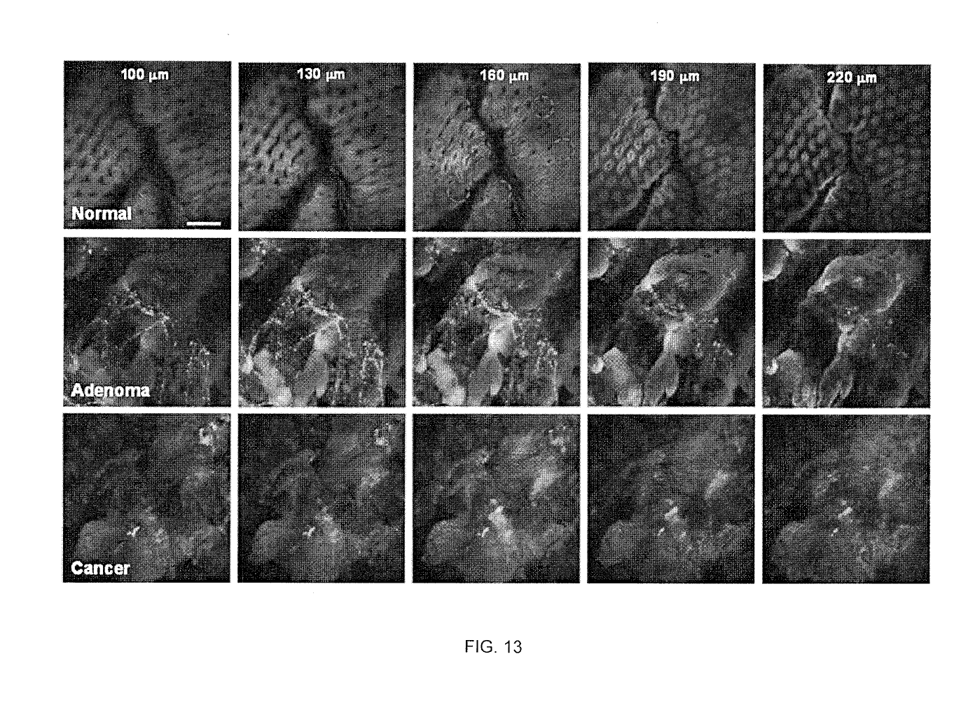

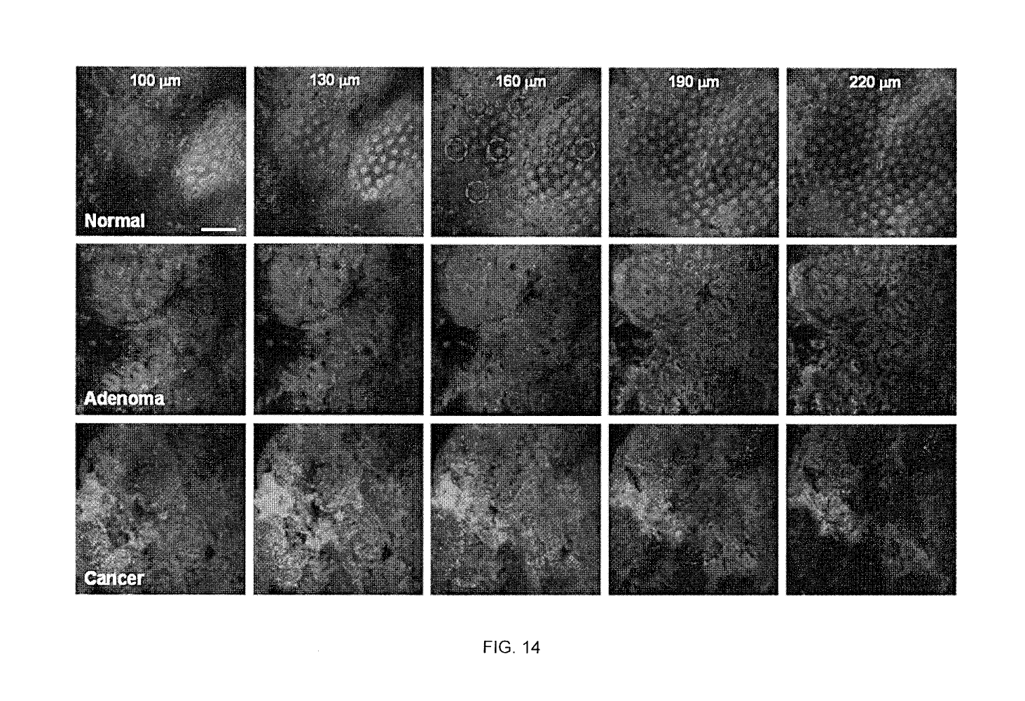

In order to determine the 3-dimensional distribution of Cu(I)/Zn(II) in colon tissue, MPEF was detected from 7-8 xy-planes at 100-200 μm depths in the z-axis. For each plane, 10 regions of interest (ROIs) were selected without bias (see FIGS. 13 and 14). FIGS. 13 and 14 show multiphoton microscopic images of normal, adenoma and cancer tissues obtained along the z-direction over the depths of 100-220 μm after labeling with the compound of Preparation Example 1 and the compound of Preparation Example 2, respectively. The ROIs were marked by white circles. TPEF was collected at 500-620 nm after excitation at 750 nm with fs pulse. The scale bars are 300 μm long and the tissues shown are representative images from replicate experiments (n=75). MPEF intensities of the 70-80 ROIs from the 7-8 xy-planes were summed and then divided by the total number of the ROIs to obtain mean MPEF intensity per ROI. This value was used as relative Cu(I)/Zn(II) level.

Statistical Analysis

Statistical analysis was performed by the Wilcoxon signed-rank test. The result was given as mean±standard deviation. Significance level was set as P<0.05.

Result

Human colon tissue was donated from the patients who were histologically diagnosed as colon cancer or colorectal adenoma. A total of 28 patients were divided into two groups. There was no significant difference between the two groups in average age, sex and basic features (see Table 1).

TABLE 1

| | | | | MPEF/Cu/ | | MPEF//Zn/ | | | | | MLH-1 | MSH-2 |
|---|---|---|---|---|---|---|---|---|---|---|---|---|
| Patient | | | Pathological | normal | | normal | MPEF/Zn/ | c-Kit | | p53 | loss | loss |
| No. | Sex | Age | condition | tissue | MPEF/Cu/tumor | tissue | tumor | (%) | EGFR | (%) | (%) | (%) |
| 1 | M | 62 | TA, LGD | 114.5 ± 39.7 | 193.8 ± 5.8 | 174.5 ± 33.1 | 84.9 ± 8.8 | NA | NA | NA | NA | NA |
| 2 | F | 61 | TA, HGD | 258.3 ± 15.4 | 487.4 ± 8.8 | 233.6 ± 12.7 | 112.3 ± 22.6 | NA | NA | NA | NA | NA |
| 3 | M | 63 | TA, LGD | 128.3 ± 17.9 | 234.9 ± 13.6 | 197.9 ± 23.8 | 111.3 ± 14.1 | NA | NA | NA | NA | NA |
| 4 | F | 76 | TA, LGD | 83.4 ± 11.9 | 201.3 ± 17.5 | 221.1 ± 11.3 | 119.2 ± 14.6 | NA | NA | NA | NA | NA |
| 5 | M | 68 | Cancer, | 104.6 ± 17.9 | 239.8 ± 4 | 153.7 ± 23.9 | 96.2 ± 20.8 | NA | NA | NA | NA | NA |
| | | | poor | | | | | | | | | |
| | | | difference | | | | | | | | | |
| | | | (T4N2M0) | | | | | | | | | |
| | | | TA, LGD | | 208.1 ± 13.5 | | 105.5 ± 15.3 | NA | NA | NA | NA | NA |
| 6 | M | 70 | Cancer, | 167.0 ± 20.2 | 245.7 ± 10.5 | 189.6 ± 39.0 | 89.2 ± 19.6 | 0 | 2+ | 80-90% | 10-20% | 70-80% |
| | | | moderate | | | | | | | | | |
| | | | difference | | | | | | | | | |
| | | | (T3N0M0) | | | | | | | | | |
| | | | TA, LGD | | 230.9 ± 5.2 | | 109.1 ± 19.4 | NA | NA | NA | NA | NA |
| 7 | M | 76 | Cancer, | 99.7 ± 11.8 | 213.1 ± 19.0 | 217.1 ± 16.9 | 120.6 ± 17.3 | <5% | 1+, | 0 | <25% | 25-50% |
| | | | moderate | | | | | | | | | |
| | | | difference | | | | | | | | | |
| | | | (T3N0M0) | | | | | | | | | |
| 8 | M | 65 | TA, LGD | 85.9 ± 15.2 | 216.8 ± 11.1 | 217.1 ± 16.9 | 120.6 ± 17.3 | NA | NA | NA | NA | NA |
| 9 | F | 55 | TA, LGD | 100.6 ± 23.5 | 173.0 ± 32.5 | 174.2 ± 17.3 | 122.7 ± 18.8 | NA | NA | NA | NA | NA |
| 10 | M | 69 | Cancer, | 108.9 ± 9.9 | 228.5 ± 14.3 | 191.4 ± 17.9 | 105.9 ± 14.2 | NA | NA | NA | NA | NA |
| | | | good | | | | | | | | | |
| | | | difference | | | | | | | | | |
| | | | (T1N0M0) | | | | | | | | | |
| 11 | M | 58 | Sawtooth-shaped | 113.3 ± 19.9 | 224.5 ± 18.9 | 190.4 ± 17.9 | 114.7 ± 14.4 | NA | NA | NA | NA | NA |
| | | | adenoma, | | | | | | | | | |
| | | | LGD | | | | | | | | | |
| 12 | F | 70 | TA, LGD | 119.7 ± 15.6 | 207.3 ± 13.3 | 174.8 ± 22.4 | 113.9 ± 16.9 | NA | NA | NA | NA | NA |
| 13 | F | 50 | Cancer, | 101.9 ± 19.6 | 193.0 ± 15.3 | 180.5 ± 12.6 | 108.9 ± 15.0 | NA | NA | NA | NA | NA |
| | | | good | | | | | | | | | |
| | | | difference | | | | | | | | | |
| | | | (T1N0M0) | | | | | | | | | |
| 16 | F | 41 | Cancer, | 95.4 ± 12.6 | 195.9 ± 22.1 | 211.1 ± 22.4 | 95.3 ± 17.6 | <5% | +3, | 95% | <5% | 50% |
| | | | moderate | | | | | | | | | |
| | | | difference | | | | | | | | | |
| | | | (T2N0M0) | | | | | | | | | |
| 17 | M | 53 | Cancer, | 94.5 ± 26.7 | 210.3 ± 24.7 | 199.6 ± 18.9 | 109.2 ± 16.5 | NA | NA | NA | NA | NA |
| | | | moderate | | | | | | | | | |
| | | | difference | | | | | | | | | |
| | | | (T4N1M0) | | | | | | | | | |
| | | | TA, LGD | | 179.9 ± 16.4 | | 101.7 ± 16.8 | NA | NA | NA | NA | NA |

The multiphoton microscopic images of the colon tissue labeled with the compound of Preparation Example 1 and the compound of Preparation Example 2 showed distinct distribution of Cu(I) and Zn(II) ions over the depths of 100-220 μm (see FIGS. 13 and 14). As seen from FIGS. 13 and 14, the normal tissue had an intact texture, but the adenoma/cancer tissue was amorphous over the whole depths. For given tissue, the 3-D distribution of Cu(I)/Zn(II) level was almost the same. But, as the tissue changed from normal state to adenoma or cancer, the Cu(I) level increased and the Zn(II) level decreased. The Cu(I)/Zn(II) ratio increased 3.5-fold from normal tissue to adenoma/cancer tissue (see Table 2).

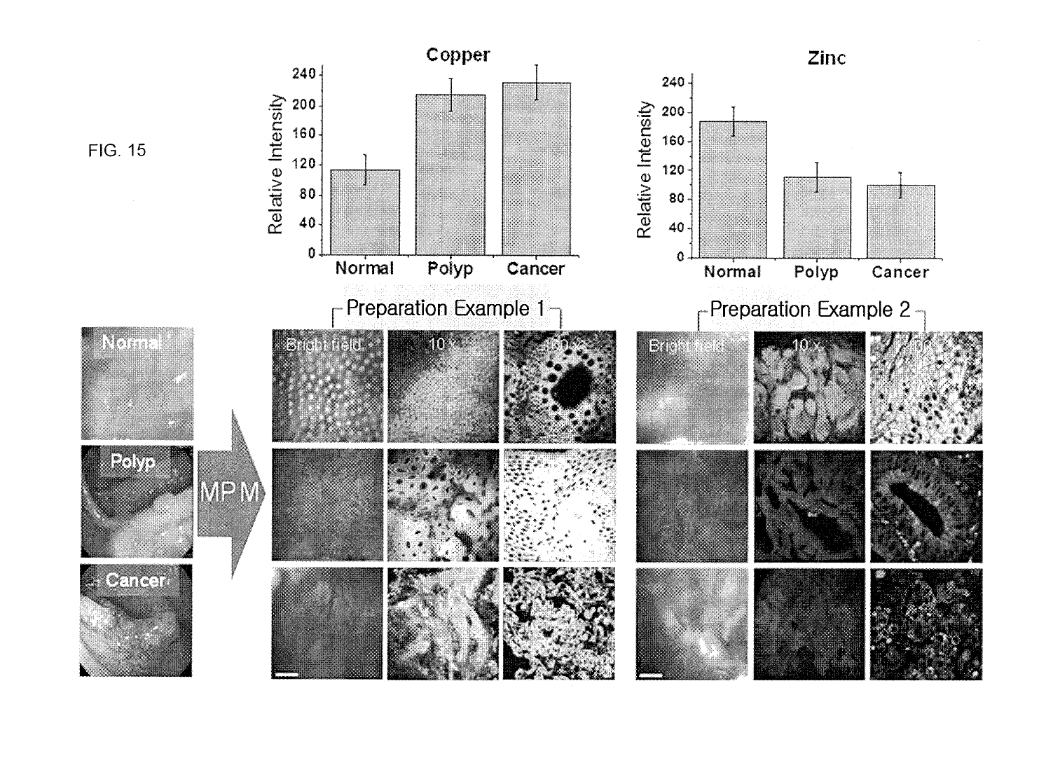

FIG. 15 shows colonoscopic images (left bottom), bright-field images and multiphoton microscopic images of normal, adenoma and cancer tissues labeled with the compound of Preparation Example 1 (20 μM, middle bottom) and the compound of Preparation Example 2 (20 μM, right bottom) obtained at a depth of 120 μm, and relative MPEF intensity of tissues labeled with the compound of Preparation Example 1 (20 μM, left top) and the compound of Preparation Example 2 (20 μM, right top). Relative MPEF intensity of the tissues labeled with the compound of Preparation Example 1 (20 μM, left top) and the compound of Preparation Example 2 (20 μM, right top) is shown above the images. TPEF was collected at 500-620 nm after excitation at 750 nm with fs pulse. The scale bars are 300 (×10) and 30 (×100) μm long and the tissues shown are representative images from replicate experiments (n=75).

TABLE 2

| | Normal mucosal tissue | Tumorous tissue | P value* |
|---|---|---|---|
| Cu(I) | 113.9 ± 20.3 | 187.9 ± 21.53 | <0.001 |
| Zn(II) | 215.5 ± 19.95 | 111.51 ± 20.26 | <0.001 |
| Cu(I)/Zn(II) | 0.551 ± 0.153 | 1.913 ± 0.256 | <0.001 |

*Cu(I), Zn(II) and Cu(I)/Zn(II) ratio were compared by the Wilcoxon signed-rank test.

### CONCLUSION

The multiphoton microscopic images of the ACT116 and HT-29 cells labeled with the compound of Preparation Example 1 was brighter than the NIH3T3 cells, and the opposite result was observed when the cells were labeled with the compound of Preparation Example 2. These results suggest that Cu(I) is more abundant in cancer cells than in normal cells and Zn(II) is more abundant in normal cells than in cancer cells. In the colon tissue, the Cu(I) level increased gradually as the tissue changed from normal state to adenoma or cancer and the Zn(II) level decreased. As a result, the Cu(I)/Zn(II) ratio increased gradually. The Cu(I)/Zn(II) ratio increased 3.5-fold from normal tissue to adenoma/cancer tissue (see Table 2). This means that a high Cu(I)/Zn(II) ratio is a useful index for diagnosing colon cancer.

The present disclosure has the following advantages over the existing techniques.

First, the multiphoton microscopic images of tissue labeled with the compounds of Preparation Example 1 and Preparation Example 2 show distinct metal ion distribution at different depths over 100-200 μm (see FIGS. 3 and 4). The normal tissue showed an intact texture, but the adenoma/cancer tissue had amorphous texture. Accordingly, considering that imaging at different depths is impossible with the existing optical or confocal microscope, the diagnosis of malignant disease based on the observation of tissue texture is possible only by the present disclosure.

Next, in accordance with the present disclosure, the difference in metal ion levels in normal tissue and malignant tissue can be easily determined by comparing multiphoton microscopic images of tissue samples. Although the cause is not clear yet, the difference in metal ion levels in the tissues may provide information on whether the tissue is healthy or not. Moreover, from comparison of tissue images at different depths, it can be determined how far the malignancy developed.

Lastly, in accordance with the present disclosure, multiphoton microscopic images can be obtained within a few hours after biopsy. The existing pathological diagnosis based on optical microscopy and hematoxylin and eosin (H&E) staining is time-consuming since the procedure of formalin fixation, paraffin embedding, slicing and staining are required. At least 2-3 days are required until the result is obtained. In contrast, in accordance with the present disclosure, time can be saved since fresh biological samples obtained from biopsy specimen can be observed directly.

The fluorescent probe according to the present disclosure is capable of detecting free copper(I) ions inside cells for a long time with high selectivity and sensitivity for copper(I) ion, with a penetration depth longer than 90 μm in living cells and tissues and without the problems of mistargeting and photobleaching. Accordingly, since a biological sample can be imaged for a long period of time with high resolution without damage, presence of malignant disease in the target biological sample can be diagnosed faster, more accurately and more easily.

While the present disclosure has been described with respect to the specific embodiments, it will be apparent to those skilled in the art that various changes and modifications may be made without departing from the spirit and scope of the disclosure as defined in the following claims.

What is claimed is:

1. A copper(I) ion-selective fluorescent probe of Chemical Formula 1: 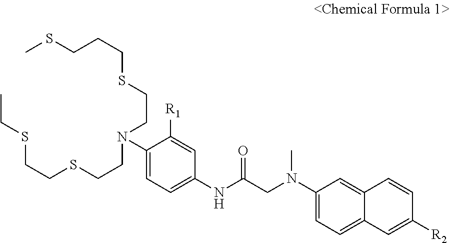
wherein R₁ is hydrogen, C₁-C₁₀ alkyl or C₁-C₁₀ alkoxy, R₂ is —COCH₃, 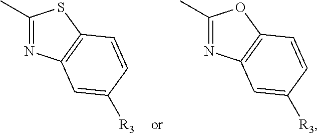
and R₃ is hydrogen or C₁-C₁₀ alkoxy.

2. The copper(I) ion-selective fluorescent probe of claim 1, wherein R₁ is hydrogen, methyl or methoxy.

3. The copper(I) ion-selective fluorescent probe of claim 1, wherein R₃ is hydrogen or methoxy.

4. The copper(I) ion-selective fluorescent probe of claim 1, wherein the compound of Chemical Formula 1 is a compound of Chemical Formula 2:

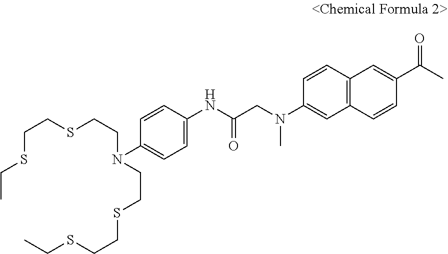 [SMILES: CCSCCSCCN(CCSCCSCC)c1ccc(NC(=O)CN(C)c2ccc3cc(C(C)=O)ccc3c2)cc1]

5. A method for preparing a copper(I) ion-selective fluorescent probe, comprising reacting a compound of Chemical Formula 3 with a mixture of 6-acetyl-2-[N-methyl-N-(carboxymethyl)amino]naphthalene, 1-hydroxybenzotriazole and N,N′-dicyclohexylcarbodiimide to prepare a compound of Chemical Formula 2:

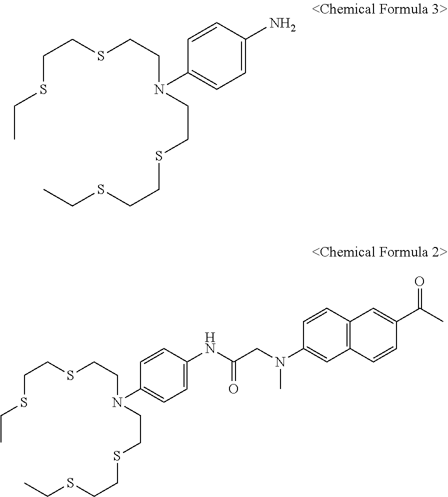 [SMILES: CCSCCSCCN(CCSCCSCC)c1ccc(N)cc1 ; CCSCCSCCN(CCSCCSCC)c1ccc(NC(=O)CN(C)c2ccc3cc(C(C)=O)ccc3c2)cc1]

6. The method for preparing a copper(I) ion-selective fluorescent probe as set forth in claim 5, wherein the compound of Chemical Formula 3 is prepared by refluxing a mixture of a compound of Chemical Formula 4 and SnCl₂ in an organic solvent:

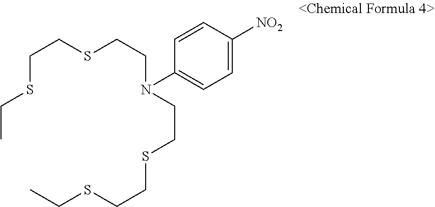 [SMILES: CCSCCSCCN(CCSCCSCC)c1ccc([N+](=O)[O-])cc1]

7. The method for preparing a copper(I) ion-selective fluorescent probe as set forth in claim 6, wherein the compound of Chemical Formula 4 is prepared by reacting a mixture of a compound of Chemical Formula 5, 2-(ethylthio)ethanethiol and Cs₂CO₃: 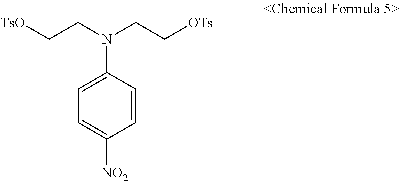
wherein Ts is tosyl.

8. A method for diagnosing malignant disease, comprising:
labeling copper(I) ions and zinc(II) ions in a biological sample respectively with a copper(I) ion-selective fluorescent probe of Chemical Formula 1 and a zinc(II) ion-selective fluorescent probe;
measuring multiphoton fluorescence intensity for the copper(I) ions and zinc(II) ions by multiphoton microscopy;
calculating the ratio of the multiphoton fluorescence intensity for the copper(I) ions to the multiphoton fluorescence intensity for the zinc(II) ions from the measured values; and
diagnosing malignant disease using the ratio: 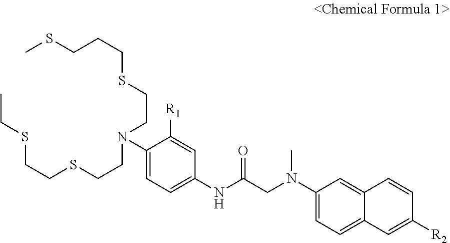
wherein R₁ is hydrogen, C₁-C₁₀ alkyl or C₁-C₁₀ alkoxy, R₂ is —COCH₃, 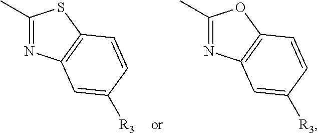
and R₃ is hydrogen or C₁-C₁₀ alkoxy.

9. The method for diagnosing malignant disease as set forth in claim 8, wherein the zinc(II) ion-selective fluorescent probe is a compound of Chemical Formula 6: 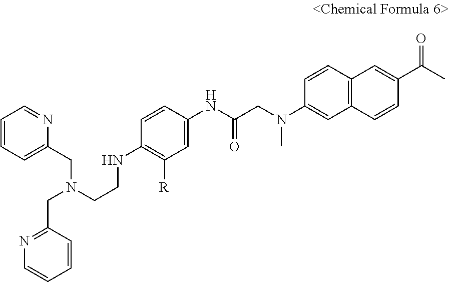
wherein R is hydrogen or OCH₃.

10. The method for diagnosing malignant disease as set forth in claim 8, wherein the malignant disease is respiratory cancer, gastrointestinal cancer or breast cancer.

11. The method for diagnosing malignant disease as set forth in claim 8, wherein the malignant disease is diagnosed when the ratio is between 1.657 and 2.169.

12. A kit for diagnosing malignant disease comprising:
a first probe-attached portion to which a copper(I) ion-selective fluorescent probe of Chemical Formula 1 is attached;
a second probe-attached portion to which a zinc(II) ion-selective fluorescent probe is attached; and
a biological sample introducing unit introducing a biological sample to the first and second probe-attached portions: 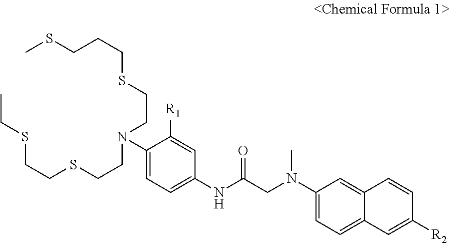
wherein R₁ is hydrogen, C₁-C₁₀ alkyl or C₁-C₁₀ alkoxy, R₂ is —COCH₃, 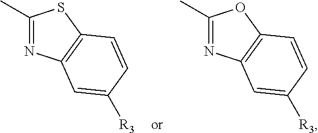
and R₃ is hydrogen or C₁-C₁₀ alkoxy.

13. The kit for diagnosing malignant disease of claim 12, wherein the zinc(II) ion-selective fluorescent probe is a compound of Chemical Formula 6: 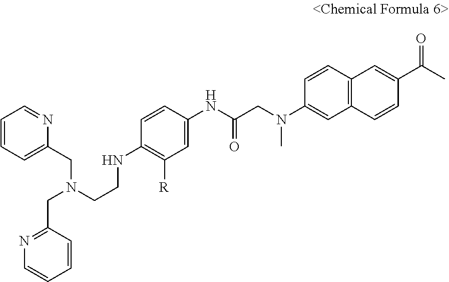
wherein R is hydrogen or OCH₃.
